(19) United States
(12) Patent Application Publication
(10) Pub. No.: US 2003/0106836 A1
(43) Pub. Date: Jun. 12, 2003

(54) Batch process for making high flash point pitch

(76) Inventor: Thomas H. Orac, Strongsville, OH (US)

Correspondence Address: Waddey & Patterson, P.C., Bank of America Plaza, 414 Union Street, Suite 2020, Nashville, TN, 37219, US

(21) Appl. No.: 10/013,072

(22) Filed: Dec. 10, 2001

(51) Int. Cl. C01C 1/04; C07C 27/10

(52) U.S. Cl. 208/43; 208/4

(57) ABSTRACT

A method for producing a high-flash-point impregnating pitch that comprises charging coal tar into a batch still; heating the coal tar to an intermediate liquid state of soft pitch; maintaining the intermediate temperature of the soft pitch at a steady level; and introducing sparging gas while maintaining the temperature of the coal tar at a substantially steady level. The method of the present invention provides a pitch that has a softening point of about 90° C. and a flash point of above about 270° C.

### DESCRIPTION

1. Technical Field of the Invention

This invention relates to the field of pitch production. More specifically, this invention is directed to a process that allows for the production of a pitch that has a softening point of about 90° C. and a flash point of higher than 270° C.

2. Background of the Art

Carbon and graphite bodies are porous and many of these products require that the baked stock be impregnated with pitch before graphitization to decrease the porosity and increase the strength of the final product. Examples of such bodies are graphite electrodes, which are used in the steel industry to melt the metals and other ingredients used to form steel in electric-arc furnaces. Pitch, such as the pitch of the present invention, is typically produced from coal derivatives such as coal tar. The production of pitch is described in, for example, the Encyclopedia of Chemical Technology, Kirk-Othmer, Volume 23, pages 679-717, the contents of which are incorporated herein by reference.

Coal tar pitches are mixtures of hundreds of chemical species. Most are straight hydrocarbons, some contain other elements such as sulphur or nitrogen. The compounds are highly aromatic (i.e., cyclic). Molecular weights vary from approximately 300 to in excess of 1,000.

A carbonized electrode is typically impregnated with liquid pitch at elevated temperature to reduce the pitch viscosity. It is also conventional to preheat the electrode to an elevated temperature before being contacted with the pitch impregnant under elevated pressure. After impregnation, the electrode is cooled to solidify the impregnant. After the pitch is impregnated into the carbonized or graphite body, the stock is typically re-baked to carbonize the impregnant.

A conventional way of making pitch is to heat a charge of coal tar in a batch still until the softening point reaches the desired value—about 90° C. for impregnating pitches, higher for various binder pitches. The final flash point of the pitch is typically between 245° C. and 265° C., as measured by the Cleveland Open Cup test.

U.S. Pat. No. 5,501,729 to Lewis discloses a pitch-based impregnant made from a method that involves forming a liquid solution of a substantially solids free pitch having a Q.I. of less than 1%.

U.S. Pat. No. 5,534,133 to Lewis discloses a continuous method for treating a liquid tar containing Q.I. solids to provide a liquid tar product having increased Q.I. concentration and, concurrently, a Q.I. free liquid tar product.

U.S. Pat. No. 4,931,162 to Romine discloses a process for producing clean distillate pitch and/or mesophase pitch for use in the production of carbon fibers. The pitch of this patent is obtained by distilling from an aromatic feed stock a distillate free from mesophase forming resins. The distillate is heated to obtain a heat soaked distillate, and further heated with an inert gas sparging to convert it to mesophase pitch.

U.S. Pat. No. 5,843,298 to Orac et al. discloses a process for converting coal tar containing Q.I. particles that produces a substantially Q.I.-free coal tar pitch and a separate, Q.I.-containing coal tar pitch.

U.S. Pat. No. 5,198,101 to Kalback discloses a process for producing a mesophase pitch that comprises heating the feedstock while passing a non-oxidative sparging gas such as nitrogen through the feedstock.

U.S. Pat. No. 5,730,949 to Romine discloses a method for producing metals containing anisotropic pitch product that included a heat soaking step preferably with a gas sparge.

U.S. Pat. No. 4,999,099 to Fu et al. discloses a process for making mesophase pitch that includes heating a carbonaceous feedstock while passing a reactive sparging gas through the feedstock.

U.S. Pat. No. 5,688,155 to Lewis et al. discloses graphite electrodes comprises of poorly graphitized pitch matrix material acting as a binder and/or an impregnant.

### BRIEF DESCRIPTION OF THE DRAWINGS

FIG. 1 is a chart showing the pitch softening points in relation to the weight % of distillate removed during the distillation process of the present invention described in Example 1.

FIG. 2 is a chart showing the pitch flash points in relation to the pitch softening points in connection with the pitch samples of the present invention described in Example 1.

### SUMMARY OF THE INVENTION

It is an object of the invention to produce a pitch that has a softening point of about 90° C. and a flash point of above about 270° C.

It is another object of the invention to provide a method of producing pitch that enables the flash point of pitch to be increased at a rate substantially higher (as much as twice as fast or more) as the softening point is increased. That is, the rate of the rise of the flash point is increased twice as fast as the rate of the rise of the softening point.

These and other objects achieved by the invention will be apparent from the specification.

One embodiment of the present invention is a method for producing pitch that comprises heating a batch until it becomes a soft pitch; maintaining the temperature of the batch of coal tar at a substantially steady level; and introducing sparging gas while maintaining the temperature of the batch at a substantially steady level. This embodiment of the invention is effective for making binder pitch, impregnant pitch, or both.

Another embodiment of the present invention is a method of producing a pitch impregnant by heating a batch of coal tar to an elevated temperature to obtain a softening point of between about 70° to 75° C.; and maintaining said temperature at a steady level while introducing a stream of steam or of a sparging gas into the batch until the batch's softening point reaches about 90° C. and the flash point is at least about 270° C. The flash point is measured by the Cleveland Open Cup test.

Furthermore, another embodiment of the present invention is a pitch (binder or impregnating pitch) for a carbon or graphite body in the form of a coal tar pitch with a softening point of about 90° C. (+/−about 6°, more preferably +/−about 2°) and a flash point of higher than about 270° C. The flash point is measured by the Cleveland Open Cup test. Finally, included within the scope of the present invention are impregnating and binder pitches prepared according to the processes of the present invention.

### DETAILED DESCRIPTION OF THE INVENTION

In the production of graphite bodies, in some instances the bodies are required to be impregnated with pitch to increase strength and density. This is true for graphite electrodes produced for the steel industry. They are used in the steel industry to melt the metals and other ingredients used to form steel in electric arc furnaces. The heat needed to melt the metals is generated by passing current through at least one electrode, usually three, and forming an arc between the electrodes and the metal.

The methods of producing pitch disclosed herein may be used for making binder pitch as well as impregnating pitch. Binder pitch is typically used to form the electrodes or other graphite from coke particles. Impregnating pitch is typically used to fill the pores in the formed and carbonized bodies. Some impregnation processes require an impregnating pitch that has a softening point of about 90° C. and a flash point higher than about 270° C. The softening point is measured by the Mettler method, ASTM D 3104-99. The flash point is measured by the Cleveland Open Cup test, ASTM D 92-98a. A higher flash point is advantageous because it improves safe operation.

As stated above, an embodiment of the present invention is a method for producing pitch that may be used as impregnating pitch. This embodiment includes heating a batch of coal tar in a batch still until it reaches the consistency of a “soft pitch” of about 70-75° C. softening point, and then maintaining the temperature of the coal tar at a steady level. Sparging steam or sparging gas is introduced at least while maintaining the temperature of the coal tar at a steady level.

Typically, coal tar is liquid at room temperature. As it is heated, usually in a batch still (conventionally a vertical cylindrical vessel, equipped with a heating coil), the lower-boiling point fractions gradually evaporate. As that process progresses, the softening point and the flash point gradually rise. The batch of coal tar becomes “soft pitch,” and eventually “hard pitch.” A soft pitch is generally a pitch having a softening point from about 40° C. to about 80° C. Soft pitch is available commercially.

Coal tar suitable for the present invention may be supplied by different manufacturers and is available on a commercial basis as a byproduct of manufacturing metallurgical coke.

Sparging is a technique that is commonly used in connection with pitch production and generally relates to a technique where the pitch is heated inside a closed vessel, while steam or other gasses are passed through the pitch.

In one embodiment of this process, in the first heating step, the batch of coal tar is first heated until it becomes a soft pitch, and heating continued until the soft pitch attains a softening point of between about 60° C. to about 85° C., preferably between about 70° C. to about 75° C. Optionally, sparging gas is introduced to the batch during this first heating step. In a preferred embodiment for this first heating step, if a sparging gas is used, the sparging gas is steam. Alternatively, the sparging gas may be an inert gas such as nitrogen.

In another preferred embodiment, the batch is heated in a vessel such as a drum or still until it obtains a temperature of about 260° to about 270° C. Once the batch has reached the desired temperature, the temperature is maintained (i.e., the temperature remains substantially constant). Heat input into the reaction vessel may be needed to compensate for heat loss and for heat of vaporization. However, the heat applied to the batch in the vessel should be controlled to maintain a substantially constant batch temperature. Preferably, the temperature is maintained at between about 255° C. to about 275° C., more preferably between about 260° C. to 270° C. Most preferably, the temperature variance is less than about 10° C.

Generally speaking, the temperature is maintained until the pitch softening point reaches the desired value, which is preferably about 90° C. plus or minus 2° C. However, one of ordinary skill in the art can obtain a different softening point to suit a desired application. Thus, the method of the present invention is not necessarily time-controlled, but may be considered more property-controlled.

During this period in which the temperature is maintained at a constant level, sparging gas is blown into the coal tar liquid. The sparging gas may be steam or an inert gas. When the sparging gas is an inert gas, any known gas used for sparging coal tar may be used. For the purpose of providing examples, the sparging gas may be nitrogen, helium, neon, argon, steam, or mixtures thereof.

In one embodiment of the process of the present invention, in the maintaining step, the temperature is maintained, preferably at between about 260° C. to about 270° C., at steady level until the softening point of the pitch in the vessel reaches about 90° C. In a preferred embodiment, the flash point of the batch of coal tar is higher than about 270° C., as measured by the Cleveland Open Cup test, when the softening point of the product in the still reaches 90° C. More preferably, the flash point is higher than about 280° C.

Under the conditions of this invention the flash point of the material in the vessel increases at a rate faster than the rate at which the softening point increases. More preferably, the flash point increases at a rate of about twice as fast as rate of the softening point when using the process of the present invention. Typically, the process of the present invention will produce a pitch with a flash point of from about 280° to about 300° C.

Another embodiment of the present invention is a method of producing pitch impregnant for a carbon or graphite body that comprises heating a batch of coal tar to a temperature to obtain a softening point of between about 70° to 75° C.; and then maintaining that temperature at a steady level while introducing a sparging gas into the batch. In this embodiment, the softening point of the coal tar reaches about 90° C. and the flash point of the coal tar reaches at least 270° C., preferably at least 280° C. As stated above, the present invention includes the resulting pitch impregnant for a carbon or graphite body with a softening point of about 90° C. and a flash point as measured by the Cleveland Open Cup test of higher than about 270° C., preferably at least 280° C.

Other features of the invention will become apparent in the course of the following examples which are given for illustration of the invention and are not intended to be limiting thereof.

### EXAMPLE 1

A series of pitch samples having successively higher softening points and higher flash points were produced from a coal tar sample in a laboratory distillation flask equipped with a heating mantle and inert gas sparging.

The tar was initially stripped to a low softening point pitch (˜80° C.) by sparging with inert gas. This pitch was then sparged while maintained at a constant 300° C. to produce a series of pitches with the softening points increasing up to 103° C.

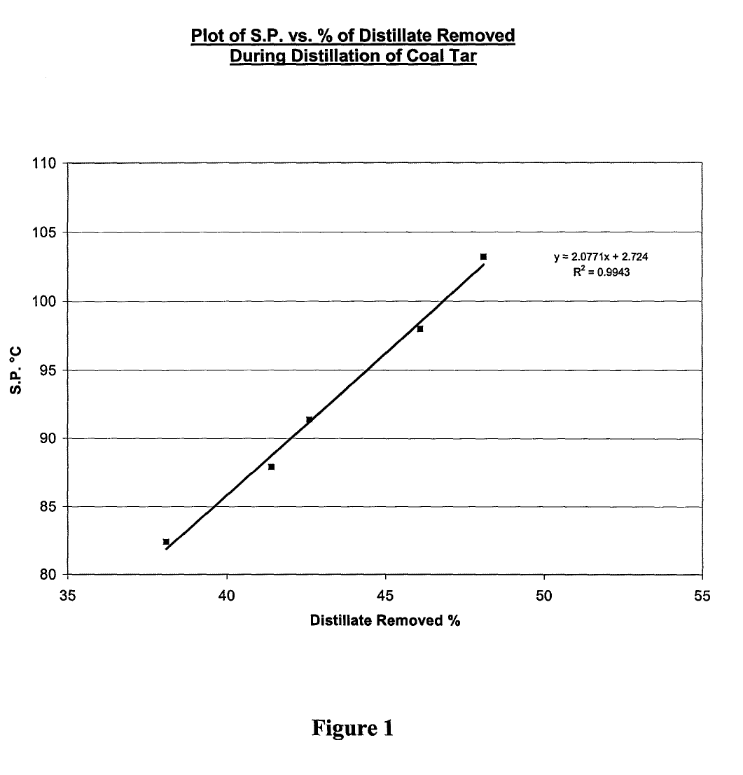

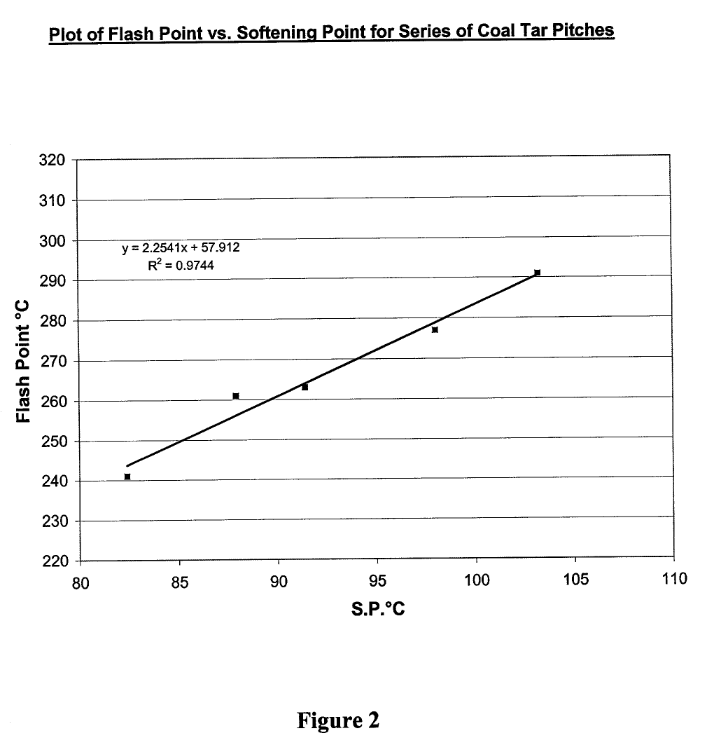

Main properties of the five pitches are summarized in Table I. The 91° C. softening point pitch was characterized more extensively, because it is close to a desired impregnation pitch; its test data are summarized in Table II. FIG. 1 is a plot of the softening point values against wt. % of distillate removed. FIG. 2 is a plot of flash points against the softening points.

The pitch softening point (“S.P”) increased linearly by 2° C. for every 1% distillate removed. The flash point increased by 2.25° C. for every degree increase in softening point.

TABLE I
Properties of Pitches Prepared from Coal Tar

| | Yield. | | | |
|---|---|---|---|---|
| Sample No. | % | Mettler S.P. ° C. | COC Flash Pt. ° C. | MCC % |
| A | 61.9 | 82.4 | 241 | 40.7 |
| B | 58.6 | 87.9 | 261 | 44.0 |
| C | 57.4 | 91.4 | 263 | 43.8 |
| D | 53.9 | 98.0 | 277 | 47.2 |
| E | 51.9 | 103.2 | 291 | 48.4 |

TABLE II
Characterization Data for “Impregnation Pitch” (Sample C)

| | |
|---|---|
| Mettler Softening Point | = 91.4° C. |
| Tg [Glass Transition Temp] | = 34° C. |
| Modified Conradson Coking Value (MCC) | = 43.8% |
| QuinolinInsolubles | = 1.5% |
| COC Flash Point . . . | = 263° C. |
| COC Fire Pt. | = 312° C. |
| Viscosity (225° C.) | = 12.5 cp |

### EXAMPLE 2

Fifty tons of a low-solids content coal tar were loaded into a vertical-drum pitch still equipped with a heating coil and a steam sparging coil. The still was heated at first without steam sparging, then with sparging, until the Mettler softening point [ASTM D 3104-99] reached about 70° C.-75° C. At that point the heat input was reduced to maintain a steady temperature, but steam sparging was continued at the same rate. Samples were taken every hour and tested for both Mettler softening point and Cleveland Open Cup flash point [ASTM D 92-98a]. When the softening point reached 90° C., the flash point was at 275° C. At that point the trial was terminated and the pitch was drained into a tank. The final product had a softening point of 94.7° C. and a flash point of 282° C. Subsequent trials achieved flash points ranging from 280° C. to 300° C.

Pitches produced previously in the same still from the same coal tar and using the conventional process of simultaneous heating and steam sparging had softening points around 90° C. and flash points around 260° C.

All patents and publications mentioned herein are hereby expressly incorporated by reference in their entirety.

The above description is intended to enable the person skilled in the art to practice the invention. It is not intended to detail all of the possible modifications and variations which will become apparent to the skilled worker upon reading the description. It is intended, however, that all such modifications and variations be included within the scope of the invention which is seen in the above description and otherwise defined by the following claims. The claims are meant to cover the indicated elements and steps in any arrangement or sequence which is effective to meet the objectives intended for the invention, unless the context specifically indicates the contrary.

What is claimed is:

1. A batch method for producing a high flash point pitch, comprising:
maintaining the temperature of a batch of soft pitch at a substantially steady level, and introducing a sparging gas while maintaining the temperature of the batch at a substantially steady level.

2. The method of claim 1, wherein the soft pitch is produced by:
(1) providing a batch of coal tar; and
(2) heating the batch of coal tar until the batch of coal tar becomes a soft pitch.

3. The method of claim 2, wherein:
in step (2), the batch of coal tar is heated until it obtains a softening point of between about 40° C. to about 80° C.

4. The method of claim 3, wherein:
in step (2), the batch of coal tar is heated until it obtains a softening point of between about 70° C. to about 75° C.

5. The method of claim 2, wherein:
in step (2), the batch of coal tar is heated until it obtains a temperature of about 2600 to about 270° C.

6. The method of claim 1, wherein the batch is maintained at a temperature of between about 255° C. and about 275° C.

7. The method of claim 1, wherein the temperature is maintained at a substantially steady level with a temperature variance of no greater than about 10° C.

8. The method of claim 1, wherein the sparging gas is steam.

9. The method of claim 1, wherein the sparging gas is an inert gas.

10. The method of claim 9, wherein the sparging gas is steam, nitrogen, argon, helium, neon, or mixtures thereof.

11. The method of claim 1, wherein the temperature is maintained at a substantially steady level until the softening point of the batch reaches about 90° C.

12. The method of claim 11, wherein the temperature is maintained until the flash point of the batch is higher than about 270° C. as measured by the Cleveland Open Cup test.

13. The method of claim 1, wherein the flash point in the batch increases at a rate faster than the rate the softening point increases.

14. The method of claim 12, wherein the flash point is from about 270° C. to about 300° C.

15. A method of producing pitch for a carbon or graphite body, comprising:
(1) providing a batch of coal tar and providing a still;
(2) charging the batch into the still;
(3) heating the batch to a temperature to obtain a softening point of between about 70° C. to 75° C.; and
(4) maintaining said temperature at a steady level while introducing a sparging gas into the batch until the Mettler softening point reaches about 90° C., and the flash point of the batch is at least about 270° C. as measured by the Cleveland Open Cup Test.

16. An impregnating or binder pitch for a carbon or graphite body having a softening point of about 84° C. to about 96° C. and a flash point as measured by the Cleveland Open Cup test of higher than about 270° C.

17. The pitch of claim 16, wherein the softening point is about 88° C. to about 92° C.

18. The pitch of claim 16, wherein the softening point is about 90° C.

19. The pitch of claim 16, wherein the flash point is from about 270° C. to about 300° C.

20. A pitch prepared according to the process of claim 1.

21. A pitch prepared according to the process of claim 2.

22. A pitch prepared according to the process of claim 6.

23. A pitch prepared according to the process of claim 11.

24. A pitch prepared according to the process of claim 12.
